(19) United States
(12) Patent Application Publication
(10) Pub. No.: US 2008/0039855 A1
(43) Pub. Date: Feb. 14, 2008

(54) Device For Delivering Bone Cement Precursors, Composites Thereof, Supporting Members, And Methods Of Using The Same

(75) Inventor: Kenneth L. Lambert, Seekonk, MA (US)

Correspondence Address: BOWDITCH & DEWEY, LLP, 311 MAIN STREET, P.O. BOX 15156, WORCESTER, MA, 01615-0156, US

(73) Assignee: Lambert Systems, L.L.C., Moose, WY (US)

(21) Appl. No.: 11/829,074

(22) Filed: Jul. 26, 2007

Related U.S. Application Data
(60) Provisional application No. 60/820,370, filed on Jul. 26, 2006.
(60) Provisional application No. 60/820,384, filed on Jul. 26, 2006.

(51) Int. Cl. A61B 17/58

(52) U.S. Cl. 606/93; 606/92

(57) ABSTRACT

A delivery device for a bone cement precursor, composites thereof, and support members is provided herein.

### CROSS-REFERENCE TO RELATED APPLICATIONS

Priority is hereby claimed to commonly-owned and co-pending U.S. Provisional Application Nos. 60/820,384 and 60/820,370, both of which were filed on Jul. 26, 2006, and both of which are incorporated herein by reference in their entirety.

### FIELD

The present disclosure is related to a delivery device and method of using, and in particular, to a delivery device for bone cement precursors and supporting members.

### BACKGROUND

Various bone cements have been used for hard and soft tissue repairs in an anatomy, such as a human anatomy, but they can be problematic due to their composition, strength, tensile characteristics, and the like. Attempts have been made to reinforce bone cements by disposing support members (e.g., scaffold materials, metal pins, plates and/or screws, and the like) at the repair site, usually prior to delivery of the bone cement precursor or in the short window after delivery and before setting. Placement of support devices into the cement after setting also has been attempted, but sometimes with catastrophic crumbling of the cement in the repair due to its brittleness. Some bone repairs have involved placing support members at a distance from the cement precursor. Other bone repairs have involved placing support members into the fracture voids prior to injection of the cement precursor, thereby allowing the support members to be incorporated into the setting cement.

In order to eliminate the possibility of cracking the set bone cement, attempts have been made to form composites by combining reinforcing materials (e.g., fibers, particles, and the like) with the bone cement precursor and/or using a support member with the bone cement. However, mixing such materials with the bone cement precursor has exacerbated the foregoing problems.

Hydroxylapatite (having the formula Ca₁₀ (PO₄)₆(OH)₂, hereinafter referred to as “HAP”), is the normal crystalline component of human bone. Human bone comprises approximately 50% by weight of HAP; and the remainder comprises water and various bioactive proteins. Collagen, which is the main constituent of the protein fraction of bone, bonds as a fiber with HAP crystals to form the robust structural unit of bone.

Synthetic HAP has been used in bone surgery, and its use is well tolerated by the body. Despite this, the use of HAP for the repair of fractures and other bony defects has been limited, because HAP is considered difficult to handle, leading to frustration for surgeons.

Some surgical techniques using HAP have involved, for example, the manual preparation of HAP in the operating room immediately prior to surgery in a mortar and pestle or in complex mixing machines that can be unreliable. The surgeon can then transfer the HAP precursor to the repair site. Transfer of the HAP precursor to the patient can be accomplished by manually applying the precursor to the repair site, or by using a delivery device.

Delivery of the HAP precursor has been attempted with devices such as manually operated syringes and/or cannulas. However, such devices were designed for injection of thin fluids rather than for viscous, non-Newtonian thixotropic mixtures such as HAP cement or its precursors. Therefore, in use, when sufficient manual pressure is applied to such devices in order to be able to dispense the viscous HAP precursor, such attempts involved phase shift of the injectant (often termed “dewatering”) of the precursor, causing incomplete delivery of an unpredictable gradient of liquid/solid ratios to the repair site. Thus, the phase of the delivered material can be different than the advertised product and can have a delivered gradient of first watery material, progressing to a more solid phase, followed by material that is too dewatered to flow, thereby plugging the delivery device. Therefore, there is no assurance that surgeons can deliver a material with sufficient strength and fracture toughness to be effective for repairs.

Whether delivered manually or using a delivery device, precise placement of the HAP precursor can be difficult, especially if it sets relatively quickly. If precise placement of the paste-like precursor is not achieved, removal of the excess can be difficult, either before or after setting. Thus, formation and delivery of HAP using various techniques often can lead to inconsistent or unreliable results due to considerable variations in the consistency of the HAP precursor, difficulties in accurately positioning the HAP at the desired site and/or delivering excessive HAP to the desired site.

Another reason for the poor acceptance of HAP by surgeons is that due to its single phase crystalline structure, it can be too brittle due to insufficient tensile properties for supporting a robust composite structure such as bone.

At least in part due to the foregoing issues, acceptance of HAP cements by orthopedic surgeons has been limited. Existing mixing and delivery systems are simply considered inadequate to provide consistent quality mixtures and accurate positioning of the HAP precursor in or at the repair site.

### SUMMARY

Disclosed herein, in one embodiment, is a mixing and delivery device for a bone cement precursor, comprising: a body comprising a receiving end and a dispensing end, the body defining a chamber fluidly connecting the receiving end and the dispensing end; a removable cover disposed at the receiving end of the body, the cover comprising an inner surface and an outer surface; and a helical mixing device removably disposed in the chamber; wherein the device is constructed and arranged to mix the bone cement precursor in the chamber, and to deliver the bone cement precursor from the chamber to a repair site.

Disclosed herein, in another embodiment, is a method of repairing tissue at a repair site, comprising: forming a bone cement precursor in a delivery device; helically dispensing the bone cement precursor from the delivery device and delivering the precursor to the repair site; and allowing the bone cement precursor to set.

Also disclosed herein is a method of repairing tissue at a repair site, comprising: forming a bone cement precursor in a delivery device; disposing a support member at the delivery device; helically discharging the bone cement precursor from the delivery device to the repair site; and allowing the bone cement precursor to set; wherein the support member is deployed when the bone cement precursor is discharged from the delivery device.

The foregoing and other features are exemplified by the following figures and detailed description.

### BRIEF DESCRIPTION OF THE DRAWINGS

Referring now to the figures, which are exemplary embodiments, and wherein like elements are numbered alike:

FIG. 1 is an exploded perspective view of an exemplary delivery device according to one embodiment of the present disclosure;

FIG. 2 is an exploded side view of the delivery device shown in FIG. 1;

FIG. 3 is an exploded side schematic view of the delivery device shown in FIG. 1;

FIG. 4 is a schematic side view of the delivery device shown in FIG. 2, in an assembled configuration;

FIG. 5 is a schematic side view of the delivery device shown in FIG. 1, showing a mixing bag disposed in the chamber;

FIG. 6 is a side view of the mixing bag shown in FIG. 9;

FIG. 7 is a bottom perspective view of the cover of the delivery device shown in FIG. 1;

FIG. 8 is a schematic view of the outer section of the cover of the delivery device shown in FIG. 1;

FIG. 9 is a top view of the cover shown in FIG. 1;

FIG. 10 is a schematic view of the cover shown in FIG. 9, through line 10-10;

FIG. 11 is a schematic side view of an alternate exemplary embodiment of a delivery device according to the present disclosure;

FIG. 12 is a side view showing the assembly of the delivery device and mixing bag;

FIG. 13 is a perspective view of the delivery device of FIG. 12, showing the method of helically twisting the mixing bag in order to dispense the precursor;

FIG. 14 is a perspective view of the delivery device of FIG. 12, showing the helical twisting of the mixing bag disposed in the chamber;

FIG. 15 is a perspective view of the delivery device of FIG. 12, showing a support member disposed in the cannula, with the precursor being delivered from the cannula and causing deployment of the support member from the cannula;

FIG. 16 is a perspective view of the delivery device of FIG. 12, showing a support member disposed on the exterior of the cannula, with the precursor being delivered from the cannula and causing deployment of the support member from the cannula; and

FIG. 17 is another exemplary embodiment of a delivery device according to the present disclosure.

Other features and advantageous aspects of the disclosure will become apparent from the detailed description of embodiments of the disclosure herein which are provided as examples. It is to be understood that various features of the embodiments can be utilized independently of other features. In other words, not every feature of each embodiment need be incorporated in a given device or a manufacturing method in practicing the present disclosure. Thus, the illustrated embodiments are intended as examples and are not to be construed as limiting.

### DETAILED DESCRIPTION

The present disclosure is directed to devices and methods for forming and delivering bone cement precursors and support members, such as a scaffold material, to a soft or hard tissue repair site. In addition to providing increasing strength and stability to a repair, use of a support member such as a mesh or scaffold can act as a retaining and/or containment member, allowing the delivery of precise quantity to a specific repair site, without any overflow, thereby preventing uncontrolled or inadvertent injection of bone cement precursor and/or insufficient or excessive flow or overflow.

As used herein, the term “bone cement precursor” means a mixture and/or admixture of materials prior to setting (i.e., hardening) to form the bone cement. The term “set,” as used herein, means a bone cement that is sufficiently hard to allow a surgeon to, for example, remove sutures from a graft disposed therein. The term “setting,” as used herein, means a bone cement in the process of becoming set. Suitable bone cements that may be used in any of the embodiments disclosed herein include, but are not limited to, HAP, polymethylmethacrylate (hereinafter “PMMA”), and the like, and combinations comprising at least one of the foregoing. HAP may be desirable because it is the normal crystalline material in bone tissue, it not recognized as a foreign material by the body, and it is incorporated into the normal repair and remodeling of the bone.

As used herein, the term “composite bone cement precursor” means a bone cement precursor prior to setting that comprises materials in addition to those materials used to form the bone cement. The materials in addition to those used to form the bone cement are referred to hereinafter as “additive materials.” Suitable additive materials include, but are not limited to, reinforcing materials (e.g., HAP fibers, microfibers, nanofibers, and/or nanoplates, and the like), suture-like materials, collagen-based materials, bioresorbable materials, bioactive materials (e.g., ceramic, glass, glass-ceramic, composites, and the like), bone augmentation materials (e.g., bone induction materials, bone conduction materials, and the like), bone morphogenic proteins (BMPs), catalysts, etc., and the like, as well as other materials which may become available to the surgeon, and combinations comprising at least one of the foregoing. The foregoing additive materials may comprise any shape, size and/or configuration, provided that the strength of the bone cement is not compromised. For example, the foregoing additive materials may comprise particles, fibers, platelets, and the like. Certain biological products are available as fibers, and may contain bone inductive agents.

As used herein, “support member” means a structure that can provide one or more functions such as support, reinforcement, containment, and the like. Suitable support members include, but are not limited to, woven and non-woven materials, scaffolds, mesh, screens, fibers, mats, membranes, screws, etc., and the like. Suitable materials for the support members include, but are not limited to, metal, ceramic, and/or the like. The support members can combine with the bone cement at molecular and gross levels as composite structures.

For ease of illustration, bone cement precursors and composite bone cement precursors both are referred to hereinafter as “precursor” or “precursors.” Formation of the bone cement precursors used in the method may be accomplished in the operating room by, for example, forming a mixture of water and a bone cement powder and optionally one or more additive materials to form a bone cement precursor that is capable of setting to form a bone cement.

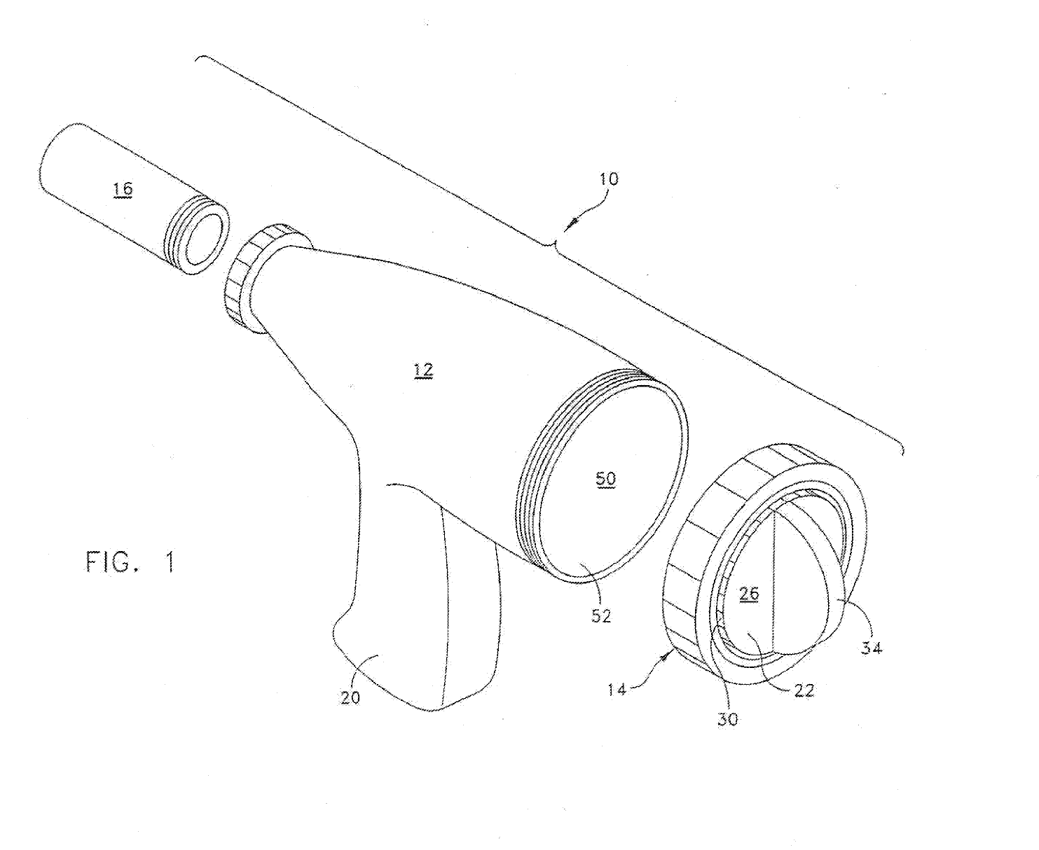

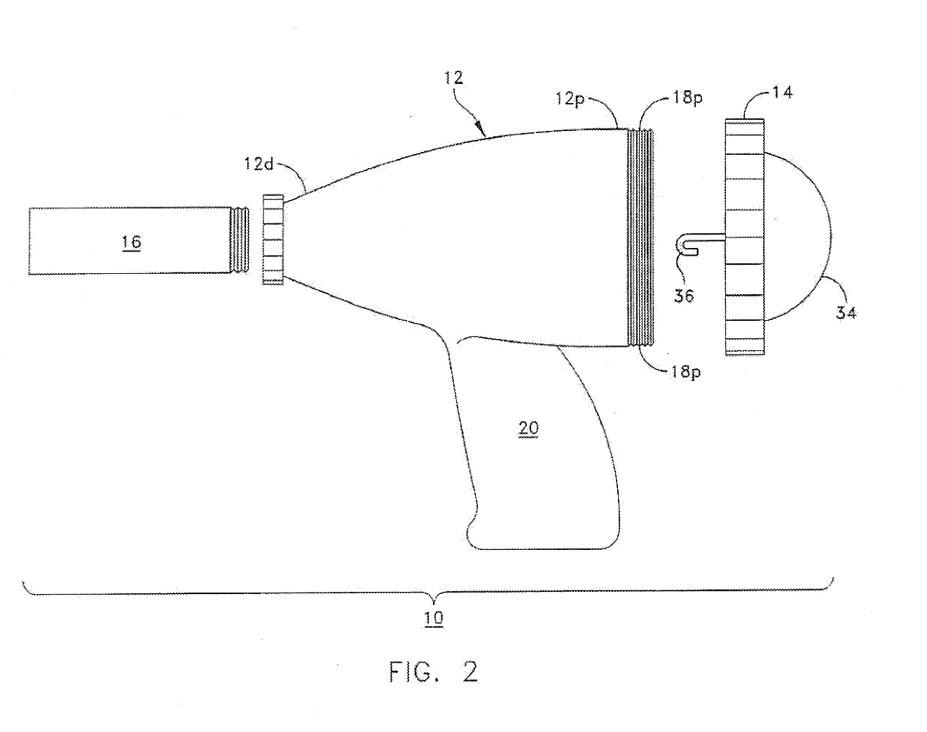

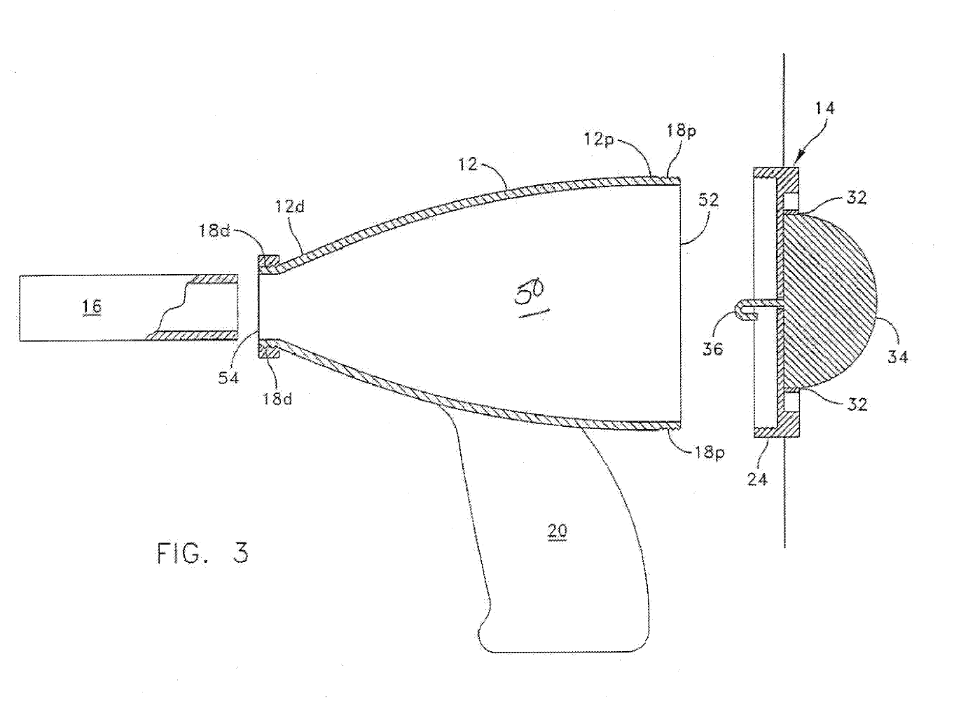

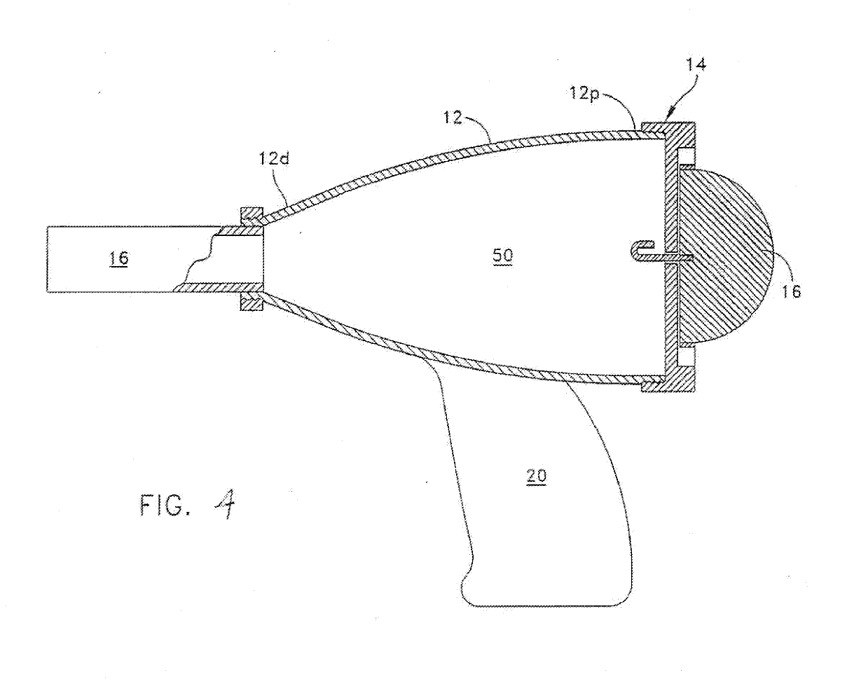

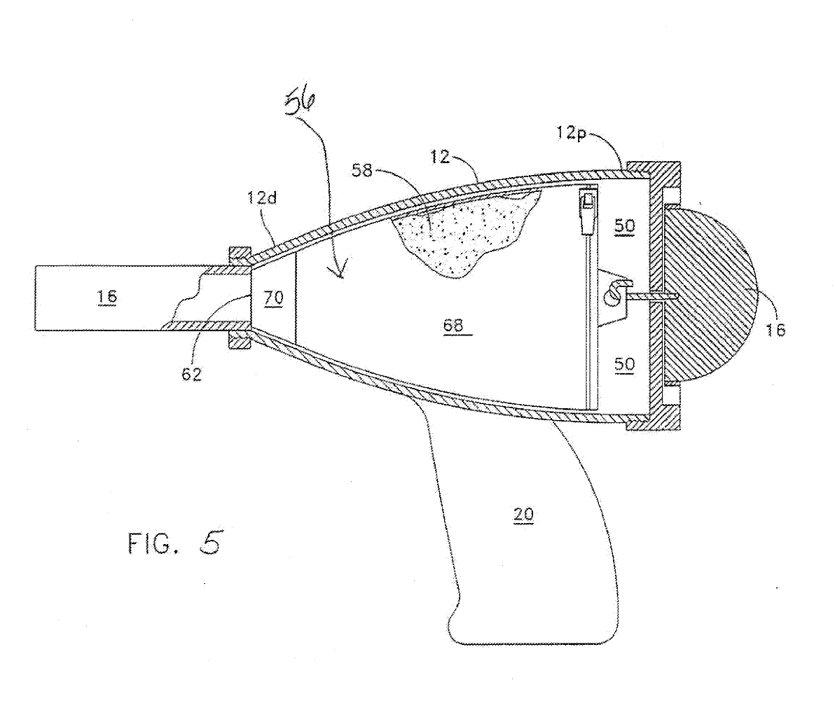

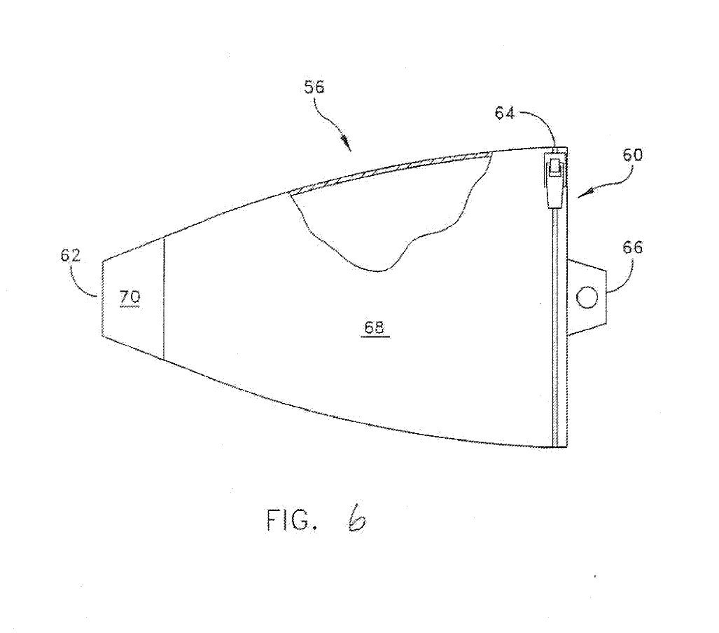

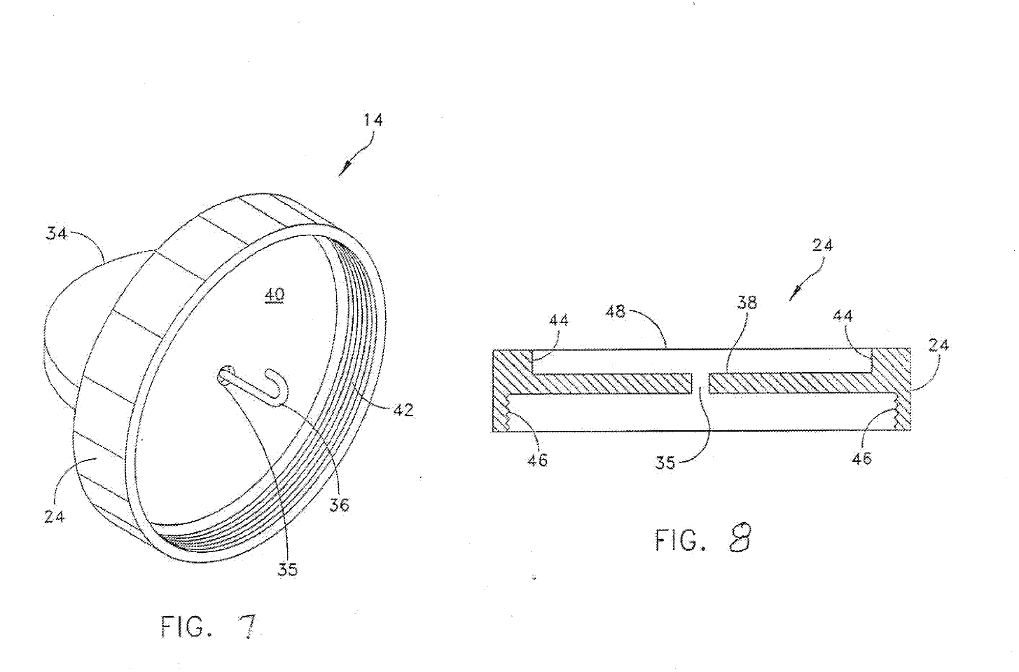

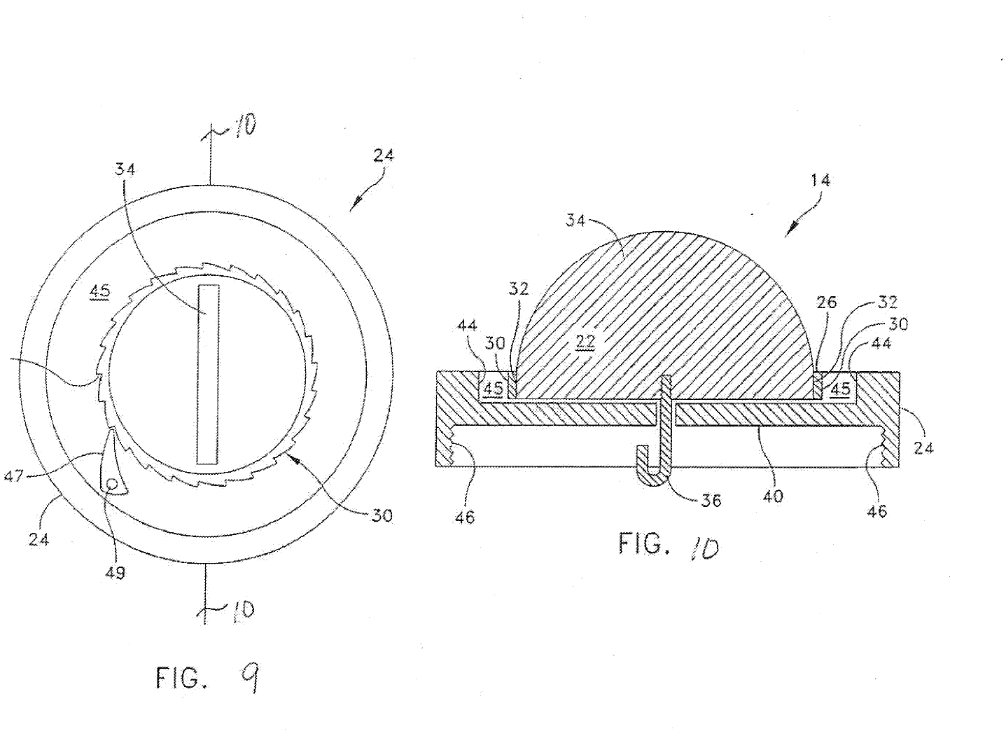

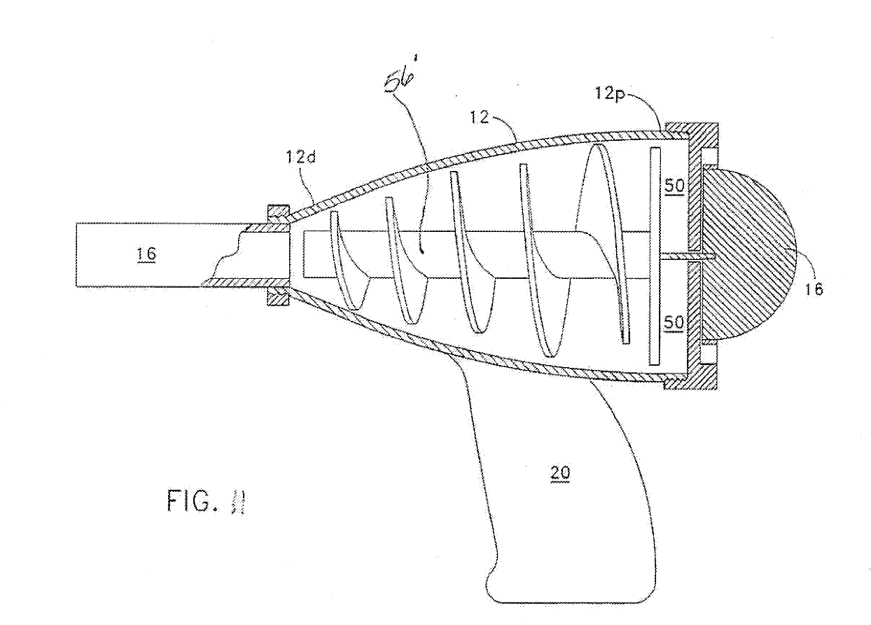

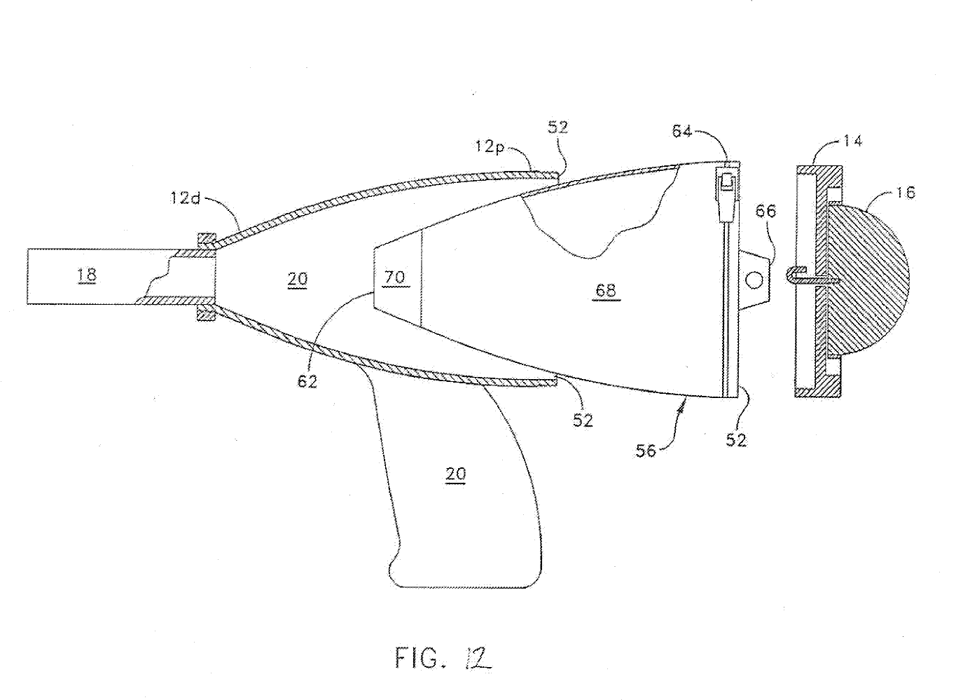

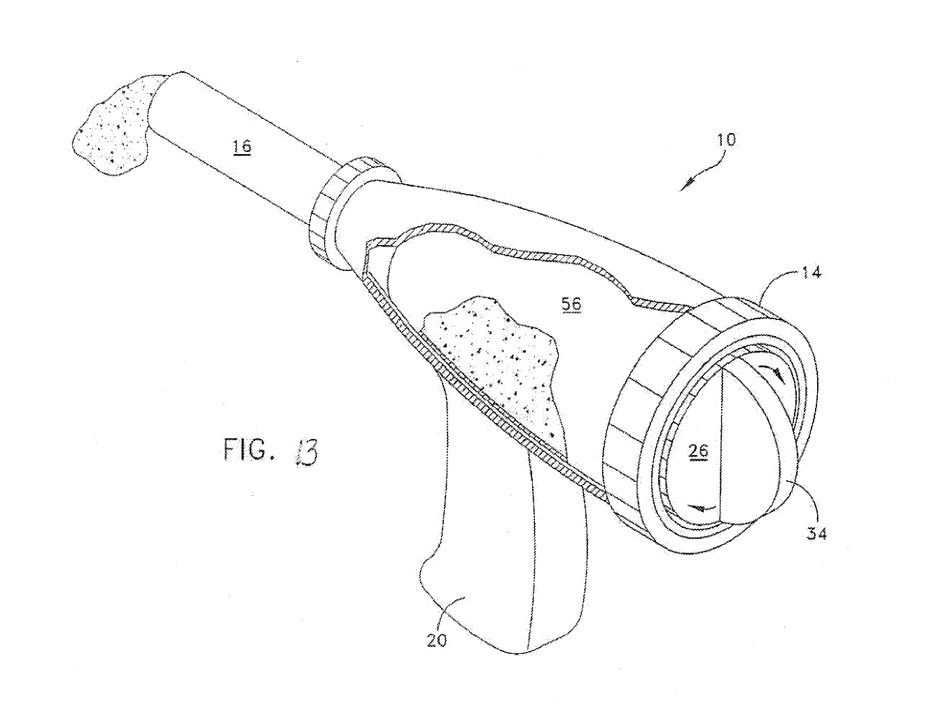

FIGS. 1-4, when taken together, show an exemplary delivery device 10 (hereinafter “device 10”) according to the present disclosure. Device 10 comprises a body 12 with opposing proximal and distal ends 12p , 12d , a removable cover 14 disposed at and supported on proximal end 12p of body 12, and optionally a removable cannula 16 disposed at distal end 12d . Body 12 also comprises externally threaded regions 18p,d disposed at each opposing end 12p , 12d . Body 12 can optionally comprise a support member 20, which is shown herein as a handle.

As shown, body 12 defines a chamber 50 fluidly connecting a proximal opening 52 at proximal end 12p and a distal opening 54 disposed at the distal end 12d . Body 12 can comprise a substantially rigid material, but other materials, including flexible materials, or a combination of rigid and flexible materials, can be used. Examples of suitable rigid materials for the chamber include but are not limited to, metal and alloys thereof, composites, phase-changing materials, and the like. Device 10 can be operated manually and/or can comprise a motor (not illustrated) and a power source (not illustrated), allowing it to be operated directly or indirectly (e.g., remotely, by wireless, robotically, automatically, and the like).

The distal end 12d of device 10 can comprise an opening 54 from which the precursor, composite precursor, support member, or combinations thereof can be dispensed for delivery directly into the patient. Thus, the need for manual transfer of a precursor from a mortar to the patient or to another device is eliminated. For example, a woven material can prevent overflow of the precursor or composite precursor into regions outside of the repair site, which can be extremely problematic if the bone cement sets before a surgeon is able to remove or clean up the “extra” bone cement.

As shown in FIG. 5, an optional removable mixing bag 56 (hereinafter “bag 56”) can be disposed in chamber 50. Bag 56 is adapted and configured to allow a precursor material 58 to be mixed in and dispensed from bag 56 and/or from distal end 12d of device 10, or from cannula 16.

As shown in FIG. 6, bag 56 comprises a receiving end 60 with an opening (not illustrated) for receiving materials, and a dispensing end 62, for dispensing the precursor after mixing in the bag. Bag 56 can be closed using a variety of techniques such as, for example, a plastic zipper 64, as shown. Also disposed at the receiving end 60 is a tab 66, which is adapted and configured for removable detachment from cover 14.

Bag 56 also comprises a first portion 68 and a second portion 70, the first portion 68 being formed from a first material that is sufficiently flexible to manipulate by hand or by twisting, without breaking. The second portion 70 can be formed from a second material that has a break strength sufficiently low such that portion 70 can break when subjected to pressure resulting from twisting bag 56, thereby allowing the mixture to be dispensed through distal opening 62. Suitable materials for bag 56 include, but are not limited to, plastic, woven materials, non-woven materials, phase-changing materials, and the like. The materials for the precursor can be introduced into the bag for mixing, either manually or automatically. The materials can be mixed to a desired viscosity and consistency in the bag. If desired, composite materials and/or support members can be introduced into the bag before, after or simultaneously with the introduction of the precursor materials into the bag.

FIGS. 7-10, when taken together, show cover 14 of delivery device 10 in greater detail. As shown, cover 14 comprises a substantially circular shape, with an inner section 22 disposed in an outer section 24. Inner section 22 comprises opposing upper and lower surfaces 26,28 spaced apart by a circumferential external surface 30 on which a plurality of ratchet gears 32 are disposed. A handle 34 is disposed on upper surface 26 of disc 22, and a hook 36 extends coaxially from the bottom surface of inner section 22, and through a central aperture 35 in outer section 24. Hook 36 can be connected or fastened to disc 22 by, for example, using a fastener such as a screw, rivet, adhesive, and the like. Handle 34 is adapted and configured to allow a user to grip and turn cover 14 during use of device 10.

Outer section 24 comprises opposing upper and lower surfaces 38, 40 and opposing downwardly and upwardly extending sidewalls 42, 44. Downwardly extending sidewall 42 comprises an internally threaded region 46 for engagement with externally threaded region 18p of body 12. Upwardly extending sidewall 44 defines a recessed region 45 in which a pawl device 47 is disposed and pivotally connected to the outer section 24 by, for example, a fastener 49 such as a rivet, screw, and the like. Pawl device 47 is adapted and configured to engage and cooperate with the corresponding ratchet gears 32 disposed on external surface 30 of coaxial disc 22, to provide substantially unidirectional movement upon turning the handle in the direction of the arrow.

FIG. 11 shows another exemplary embodiment of device 10, comprising a vane assembly 56′ extending from the lower surface of the cover. When assembled, vane assembly 56′ can be disposed in chamber 50 in order to facilitate mixing and/or delivery of the precursor. For example, the vane assembly 56′ can be disposed in chamber, adapted to operate in a first orientation in which the liquid and solids disposed in the chamber can be mixed and sheared, and in a second orientation in which the precursor can be augered from the chamber. The vanes can be concentric or eccentric, single or double. Alternatively, a piston (not illustrated) can be disposed in the chamber to facilitate mixing and/or movement of the precursor from the chamber to the receiving end in order to deliver the precursor to the patient. Other types of devices for mixing and/or moving the precursor toward the distal end of the chamber will be known to those of ordinary skill in the art.

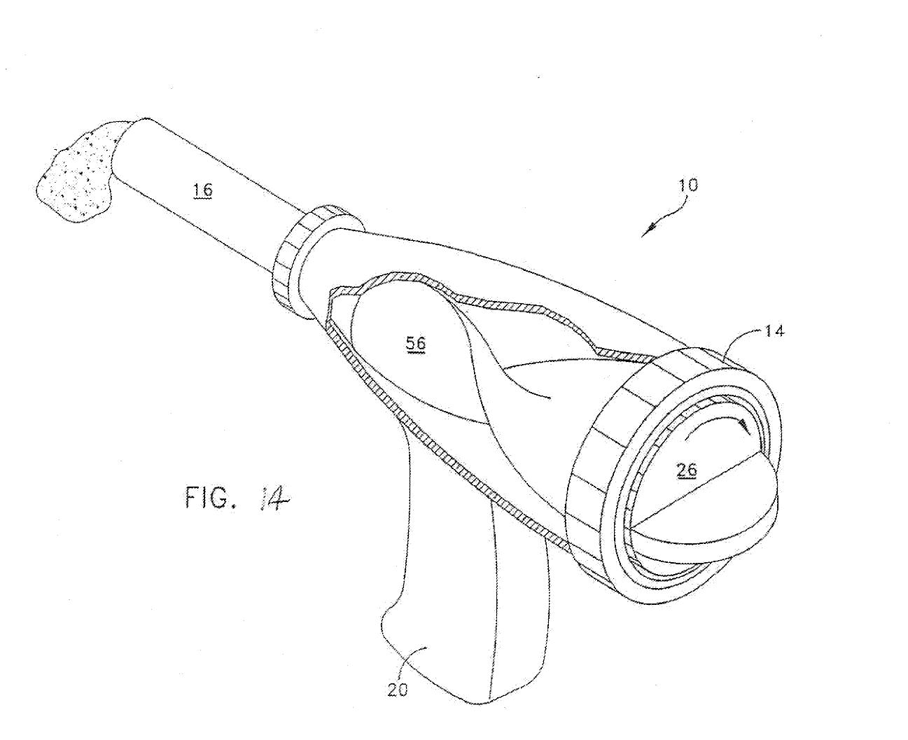

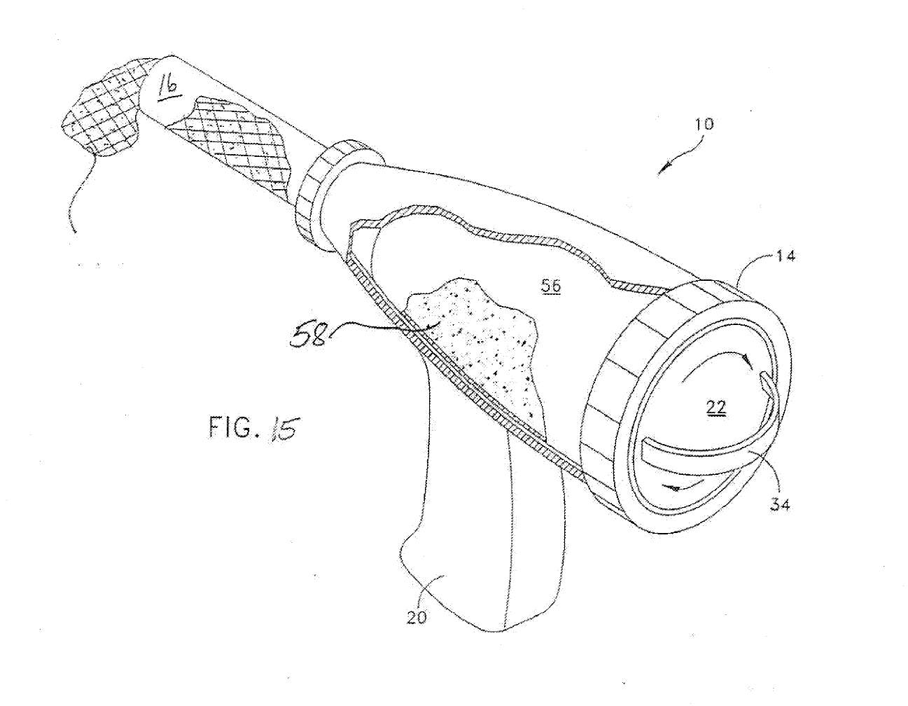

The foregoing devices facilitate a method of using the device to delivery a precursor to a repair site, as shown in FIGS. 12-14. In operation, the cover is removed from the opening at the receiving end, and the desired materials can be introduced into the chamber. After introduction of the materials into the chamber, the precursor can be mixed by, for example, kneading, rolling and/or agitating the component materials disposed in the chamber, for example using an auger as shown in FIG. 11. This can be accomplished in various manners, depending on the construction of the device. When the chamber is flexible, lined with a flexible materials, or a portion of the chamber is flexible, forming the precursor can be accomplished manually by manually kneading, rolling and/or agitating the chamber.

In use, after the precursor has been formed, it can be delivered to the repair site in the patient through the distal end of the device, for example, by reversing the orientation of the vane assembly, or via a piston or plunger. If the chamber is flexible or a portion of the chamber is flexible, the precursor can be extruded from the device by helical constriction, i.e. by twisting. Extrusion of material by helical constriction can mimic the body's natural means of mixing and moving thickened materials e.g., blood, feces, etc. The use of helical constriction to extrude the precursor allows delivery of the precursor at a relatively low pressure and in addition can progressively improve the ratio of the diameters of the chamber and cannula, thereby avoiding the pressure drop where most dewatering occurs.

If it is desired to use composite materials, the additive materials for the composite bone cement precursor can be combined with the materials for the bone cement precursor simultaneously with or after mixing the bone cement precursor, either by adding the composite materials directly to the chamber and/or entraining the composite materials into the delivery flow from inside the chamber.

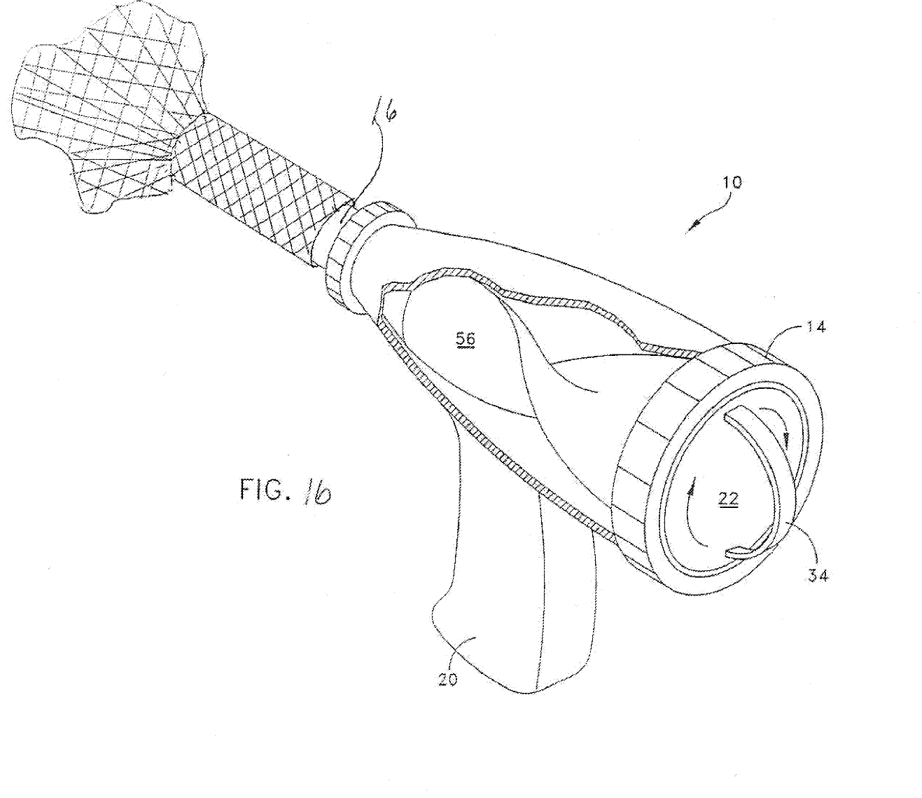

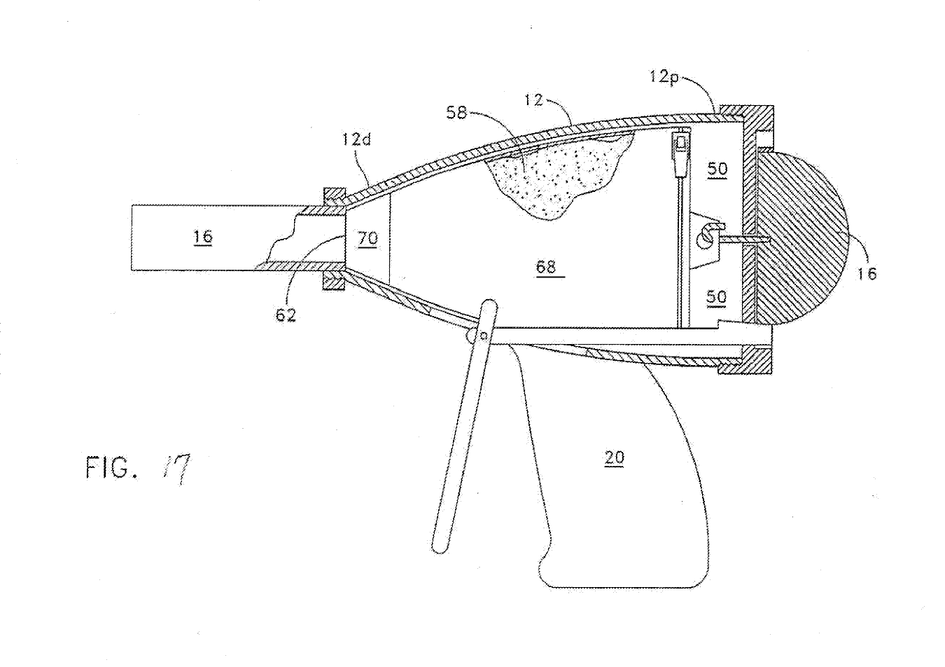

As shown in FIGS. 15 and 16, if it is desired to use support members, they can be disposed in the chamber, in the cannula and/or on the exterior surface of the cannula, and deployed therefrom by the flow of the precursor from the distal end of the device and/or from the cannula, by mechanical twisting drivers, or other energy sources all for the task of opening a flat two dimensional (“2D”) structure into a 3D scaffold. Those of skill in the art will recognize other types of devices that can be used to deploy the support members. For example, a support member comprising a sheath of fibers, mats etc., can be delivered from the chamber and/or launched from inside or outside of the outside of the cannula, for example, which can be joined in controlled quantities and spatial relationships in the filled void. The support member can be deployed from the chamber and/or from the exterior of the cannula as, for example, thin folded membranes, woven mats, three dimensional (“3D”) spatial arrays, and the like.

After deployment of a support member, it can be fixated to the walls of the void by fasteners such as screws which also can originate from the extra-cannular site of deployment. The amount and position of a support member can be controlled by its density as well as other mechanical and sonic means.

After satisfactory fill of the precursor and the support member, any portion of the support member that is remaining can be removed, e.g., by snipping with surgical scissors, thereby leaving the precursor and support member in place to set and become of adequate strength and work of fracture, providing immediate postoperative full weight bearing, and allowing use of the fractured limb or bone before natural bone healing has been completed. The deployment and the physical combining of the materials will benefit by vibration of proper frequency or frequencies to enhance the bone penetration and the critical interface between the materials.

The devices can be designed to provide a helical deployment of the support member, because this geometry typically provides the strongest construct for a given combination of composite materials.

If desired, as shown in FIGS. 13-15, device 10 can comprise a vibration source 80, which can be part of or separate from device 10. During operation of device 10, use of vibration source 80 can enhance the flow of the viscous and thixotropic precursors, the penetration of the precursors into microvoids of the bone, and the second phase material bone penetration, by providing induced shear thinning for the precursor. Vibration source 80 can be used during mixing or after delivery of the precursor, during or after injection of the precursor at the repair site, or both. The use of vibration can improve the penetration of the precursor into the small pores or interstices of the bone prior to setting, which increases the contact area between the precursor and the bone.

In one embodiment, vibration source 80 can comprise an electromagnetic coil (not illustrated) and magnet (not illustrated) driven by a tone generator (not illustrated). The vibration source can be adapted to provide a frequency range of about 1 hertz (Hz) to about ultrasonic as controlled wave forms, pulses and patterns. Optimal frequencies for mixing and delivery can be determined using no more than routine experimentation.

When separate from device 10, vibration source 80 can be disposed adjacent to and in contact with a portion of device 10 in order to provide vibrations to the precursor in the chamber and/or bag. Alternatively, vibration source 80 can be disposed adjacent to and/or in contact with the precursor after it has been disposed at the repair site, which improves penetration of precursor into the small pores or interstices of the bone prior to setting.

Vibration source 80 also can be configured and adapted to provide concussive waves, which can be used to move the thixotropic precursor. The phenomenon of concussive waves can be observed, for example, in dispensing ketchup from bottles, in the use of explosives to trigger avalanches, and at construction sites where a worker delivers intermittent blows by sledge hammer to concrete forms, and the like.

Vibration source 80 also can be configured and adapted to provide one or more hammer-like blows by adjusting the frequency and amplitude of a mass in the moving electromagnetic field, similar to a speaker coil and magnet responsive to a wide range of frequencies from 1 Hz to megahertz (MHz ), depending on the optimal frequency for a given materials flow control to be delivered, penetrated, oriented, mixed with other phase materials, control of setting by sonic energy including temperature control. For example, an ultrasound transducer may deliver this type of energy, and may be used to judge the state of the material set by ultrasonic imaging.

As used herein, the terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item. Compounds are described herein using standard nomenclature. Unless defined otherwise, technical and scientific terms used herein have the same meaning as is commonly understood by one of skill in the art to which this disclosure belongs. The modifier “about” used in connection with a quantity is inclusive of the stated value and has the meaning dictated by the context (e.g., includes the degree of error associated with measurement of the particular quantity). The terms “bottom” and “top,” and “upper” and “lower” are used herein, unless otherwise noted, merely for convenience of description, and are not limited to any one position or spatial orientation.

The device and method of the present disclosure can be advantageous because: 1) the bone cement precursor can be produced reliably and consistently; 2) the step of transferring the bone cement precursor to a delivery device can be eliminated; 3) the delivery of the bone cement precursor to the repair site can be controlled; 4) the placement of the bone cement precursor in the patient at the repair site can be controlled; 5) bio-active substances can be delivered to the repair site; 6) they provide a consistent and uniform flow of precursor, without dewatering; 7) they allow a precursor to be delivered without overflow; and 8) they eliminate the need for mixing the precursor in a separate container; and 9) they provide stability while inducing healing, especially when used with composite materials.

While the disclosure has been described with reference to an exemplary embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

What is claimed is:

1. A mixing and delivery device for a bone cement precursor, comprising:
a body comprising a receiving end and a dispensing end, the body defining a chamber fluidly connecting the receiving end and the dispensing end;
a removable cover disposed at the receiving end of the body, the cover comprising an inner surface and an outer surface; and
a helical mixing device removably disposed in the chamber; and
wherein the device is constructed and arranged to mix the bone cement precursor in the chamber, and to deliver the bone cement precursor from the chamber to a repair site.

2. The delivery device of claim 1, wherein the helical mixing device comprises a flexible bag removably connected to the cover.

3. The delivery device of claim 2, wherein the flexible bag comprises a first section constructed of a first material and a second section constructed of a second material, wherein the second section is constructed and arranged to break under a selected amount of pressure.

4. The delivery device of claim 1, wherein the helical mixing device comprises an auger removably connected to the cover.

5. The delivery device of claim 1, further comprising a cannula removably connected to the dispensing end of the body.

6. The delivery device of claim 1, wherein the mixing device is constructed and arranged to mix composite bone cement precursors.

7. The delivery device of claim 1, wherein the device is constructed and arranged to deploy a support member from the device.

8. A method of repairing tissue at a repair site, comprising:
forming a bone cement precursor in a delivery device;
helically dispensing the bone cement precursor from the delivery device and delivering the precursor to the repair site; and
allowing the bone cement precursor to set.

9. The method of claim 7, wherein the delivery device comprises a mixing chamber, and further comprising disposing the support member in the chamber.

10. The method of claim 7, wherein the delivery device comprises a cannula, and further comprising disposing the support member in the cannula.

11. The method of claim 7, wherein the delivery device comprises a cannula with an exterior surface, and further comprising disposing the support member on the exterior of the cannula.

12. The method of claim 8, wherein the support member is deployed from the chamber when the bone cement precursor is discharged from the delivery device.

13. The method of claim 9, wherein the support member is deployed from the cannula when the bone cement precursor is discharged from the delivery device.

14. The method of claim 10, wherein the support member is deployed from the exterior surface of the cannula when the bone cement precursor is discharged from the delivery device.

15. The method of claim 7, wherein the bone cement is selected from the group consisting of HAP, PMMA, and combinations comprising at least one of the foregoing.

16. A method of repairing tissue at a repair site, comprising:
forming a bone cement precursor in a delivery device;
disposing a support member at the delivery device;
helically discharging the bone cement precursor from the delivery device to the repair site; and
allowing the bone cement precursor to set;
wherein the support member is deployed when the bone cement precursor is discharged from the delivery device.

17. The method of claim 16, wherein the delivery device comprises a mixing chamber, and further comprising disposing the support member in the chamber.

18. The method of claim 16, wherein the delivery device comprises a cannula, and further comprising disposing the support member in the cannula.

19. The method of claim 16, wherein the delivery device comprises a cannula with an exterior surface, and further comprising disposing the support member on the exterior of the cannula.

20. The method of claim 17, wherein the support member is deployed from the mixing chamber when the bone cement precursor is discharged from the delivery device.

20. The method of claim 18, wherein the support member is deployed from the cannula when the bone cement precursor is discharged from the delivery device.

21. The method of claim 19, wherein the support member is deployed from the exterior surface of the cannula when the bone cement precursor is discharged from the delivery device.
